(19) United States
(12) Patent Application Publication
(10) Pub. No.: US 2021/0171996 A1
(43) Pub. Date: Jun. 10, 2021

(54) PROCESS FOR ENANTIOSELECTIVE ENZYMATIC REDUCTION OF KETO COMPOUNDS

(71) Applicant: DR. REDDY'S LABORATORIES LIMITED, Hyderabad (IN)

(72) Inventors: Shahed HUSSAIN, Hägerston (SE); Matthew BYCROFT, Cambs (GB)

(21) Appl. No.: 16/760,361

(22) PCT Filed: Oct. 31, 2018
(86) PCT No.: PCT/IB2018/058518

§ 371 Date: Apr. 29, 2020

(30) Foreign Application Priority Data
Oct. 31, 2017 (IN) 201741038693

(51) Int. Cl. C12P 19/62; C12P 17/04; C12N 9/04

(52) U.S. Cl. CPC C12P 19/62; C12N 9/0006; C12P 17/04

(57) ABSTRACT

The present application relates to enantioselective enzymatic reduction of keto compounds to the corresponding chiral hydroxy compounds. Specifically the present application describes enantioselective enzymatic reduction of ethyl-4-chloroacetoacetate (compound of formula I) into (R)-ethyl-4-chloro-3-hydroxybutyrate (compound of formula II). The present application also covers use of (R)-ethyl-4-chloro-3-hydroxybutyrate prepared by the enantioselective enzymatic reduction process in the preparation of SGLT2 inhibitor empaglifiozin.

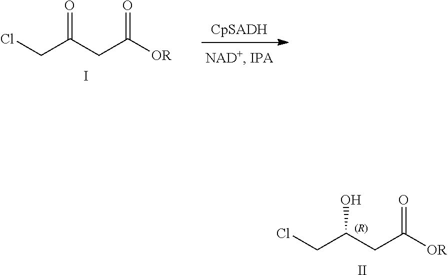

### CROSS-REFERENCE TO RELATED APPLICATIONS

This application is a National Stage Application under 35 U.S.C. § 371 of PCT International Application No. PCT/IB2018/058518, filed Oct. 31 2018 which claims the benefit of Indian provisional patent application No. 201741038693 filed on Oct. 31, 2017, all of which are herein incorporated by reference in their entireties.

### FIELD OF THE INVENTION

The present application relates to enantioselective enzymatic reduction of keto compounds to the corresponding chiral hydroxy compounds.

### BACKGROUND OF THE INVENTION

Optically active hydroxy compounds are valuable chirons with broad applicability for the synthesis of pharmacologically active compounds. The asymmetric reduction of prochiral keto compounds is a branch of stereoselective catalysis, wherein biocatalysis constitutes a powerful competitive technology versus chemical catalysis. The chemical asymmetric hydrogenation requires the use of highly toxic and environmentally harmful heavy metal catalysts, of extreme and thus energy intensive reaction conditions as well as large amounts of organic solvents. Furthermore, these methods are often characterized by side reactions and insufficient enantiomeric excesses.

Carbonyl reductases (also called alcohol dehydrogenases, oxidoreductases) are known as catalysts for the reduction of carbonyl compounds and for the oxidation of secondary alcohols, respectively. Said enzymes require a coenzyme, e.g., NAD(P)H. The reduction of ketones with the carbonyl reductase obtained from Candida parapsilosis (designated as CpSADH) and with the coenzyme NADH is known, for example, as described in Biosci. Biotechnol. Biochem., 63 (6), 1051-1055, (1999).

(R)-4-chloro-3-hydroxybutyric acid esters are, for example, important raw materials for the synthesis of SGLT-2 inhibitors. In an industrial enzymatic reduction process, said enzymes thus have to be coupled to a further enzyme responsible for the regeneration of the cofactor NADH or NADPH, respectively. Such enzymes suitable for the regeneration of NAD(P)H are formate dehydrogenase, glucose dehydrogenase, malate dehydrogenase and alcohol dehydrogenase, which preferably expressed together with the enzyme for the reduction of 4-chloro butyric acid esters.

The prior art methods fail to achieve high substrate loading and high enantiomeric excess. The present application aims at overcoming said disadvantages and relates to a process for the enantioselective enzymatic reduction of keto compounds with high substrate loading and high enantiomeric excess.

### SUMMARY OF THE INVENTION

The present application generally relates to enantioselective enzymatic reduction of keto compounds to the corresponding chiral hydroxy compounds.

In the first aspect, the present application provides a process for enantioselective enzymatic reduction of keto compound of formula I

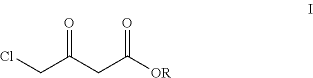

in which R represents a hydrogen atom or a C₁-C₅-alkyl group,
comprising;
treating a mixture comprising:
(a) an (R)-specific oxidoreductase obtained from Candida parapsilosis in the range of about 5 mg to about 50 mg per gram of compound of formula I,
(b) a buffer having pH in the range of 6 to 7, and
(c) 2-propanol
   with the compound of formula I in the presence of a cofactor and zinc to form a chiral hydroxy compound of general formula II

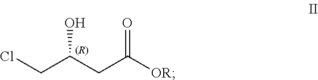

wherein, the compound of formula I in the range of about 20 to about 100 g/L is added in to the reaction mixture gradually over the course of the reaction during the production process.

In another aspect the present application provides a process for isolation of CpADH enzyme, comprising:
(a) lysing the cells expressing the enzyme CpADH by high pressure homogenization at a pH of 6 to 7 to obtain a crude extract,
(b) adding zinc sulphate or zinc chloride to the crude extract,
(c) optionally the material is clarified by the addition of polyethyleneimine, and
(d) the clear solution is dried using a lyophilizer to obtain the CpADH enzyme.

In another aspect the present application provides use of compound of formula II prepared by the process described in the present application for the preparation of pharmaceutical active ingredients.

### DETAILED DESCRIPTION

In the first aspect, the present application provides a process for enantioselective enzymatic reduction of keto compound of formula I

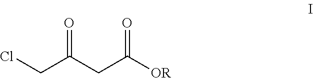

in which R represents a hydrogen atom or a C₁₋₅ alkyl group,
comprising;
treating a mixture comprising:
(a) an (R)-specific oxidoreductase obtained from Candida parapsilosis in the range of about 5 mg to about 50 mg per gram of compound of formula I,
(b) a buffer having pH in the range of 6 to 7, and
(c) 2-propanol,
   with the compound of formula I in the presence of a cofactor and zinc to form a chiral hydroxy compound of general formula II

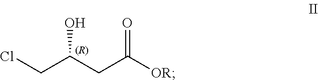

wherein, the compound of formula I in the range of about 20 to about 100 g/L is added in to the reaction mixture gradually over the course of the reaction during the production process.

The term “—C₁-C₅-alkyl” means a hydrocarbon moiety whose carbon chain is linear chain or branched and contains 1 to 5 carbon atoms, for example, methyl, ethyl, propyl, isopropyl, butyl or tertiary butyl.

The (R)-specific oxidoreductase used according to the invention is preferably from the yeast family, particularly of the genera Candida parapsilosis.

The compound of formula I is ethyl-4-chloroacetoacetate. NAD⁺ is preferably used as the cofactor. The term NAD refers to nicotinamide adenine dinucleotide.

A buffer, e.g., a potassium phosphate buffer having a pH value of 5 to 8, preferably a pH value of 6 to 7, can be added to the water. In addition, the buffer may contain metal ions for stabilizing the enzyme. In one aspect, the enzyme is stabilized by adding aqueous zinc sulphate solution or zinc chloride solution.

According to one of the aspect, a further stabilizer of alcohol dehydrogenase such as, for example, glycerol, sorbitol or dimethyl sulfoxide can be used in the process.

The temperature ranges, for example, from approximately 10° C. to 50° C., preferably from 20° C. to 35° C.

The buffer, the enzyme and the cofactor (NAD⁺) are charged into a reactor. The pH of the reaction mixture may be readjusted to 6 to 7 with a base such as potassium carbonate. A substrate solution may be prepared by dissolving the substrate (compound of formula I) in isopropanol. The substrate loading may be in the range of about 1 g/L to about 100 g/L. In one aspect the substrate loading is in the range of about 35 g/L to about 45 g/L. The substrate solution may be added slowly to the reactor over a period of about 10 minutes to about 24 hours. The process of the present invention involves adding the substrate into the reaction mixture gradually over the course of the reaction and the zinc is supplemented during the production process of the enzyme catalyst. Dosing or adding the substrate into the reaction mixture slowly over the course of the biotransformation allows a higher substrate loading to be achieved, with a loading of up to 50 g/L with complete conversion of the substrate. The completion of the reaction can be known by GC-FID method.

After completion of the reaction the reaction mixture may be diluted with an organic solvent such as diethyl ether, tetrahydrofuran or methyl tert-butyl ether. The aqueous layer may be extracted with the same solvent and the combined organic layer may be concentrated to get the compound of formula II.

In another aspect the present application provides a process for isolation of CpADH enzyme, comprising:
(a) lysing the cells expressing the enzyme CpADH by high pressure homogenization at a pH of 6 to 7 to obtain a crude extract,
(b) adding zinc sulphate or zinc chloride to the crude extract,
(c) optionally the material is clarified by the addition of polyethyleneimine, and
(d) the clear solution is dried using a lyophilizer to obtain the CpADH enzyme.

In another aspect the present application provides use of compound of formula II prepared by the process described in the present application for the preparation of pharmaceutical active ingredients.

The compound of formula II is further reduced using a suitable reducing agent such as LiAlH₄ or NaBH₄ to give a compound of formula III. The compound of formula III is cyclized using a suitable acid or a suitable base to form compound of formula IV. The compound of formula IV may be converted into a compound of formula V. The process is schematically represented below:

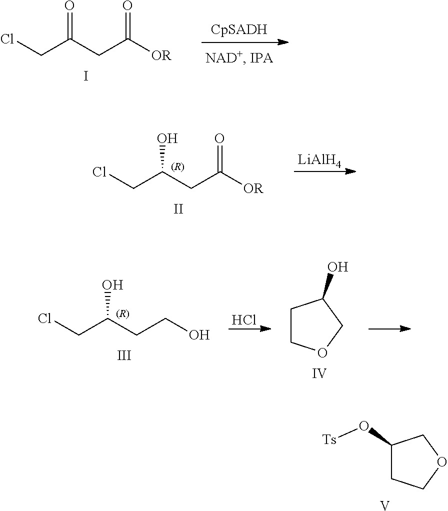

In another aspect the present application provides use of compound of formula IV or compound of formula V prepared by the process described in the present application for the preparation of an SGLT2 inhibitor empagliflozin.

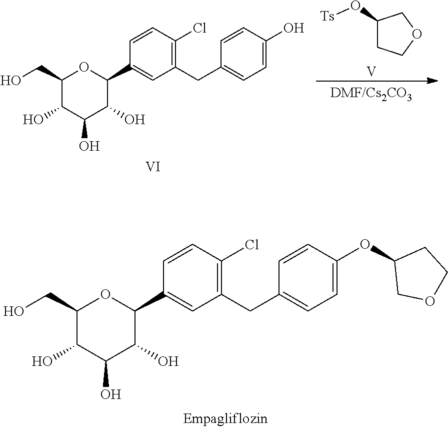

Preferred embodiments of the invention are illustrated in further detail by means of the following examples.

GC-FID Analytical Method

| | |
|---|---|
| Column | Machery-Nagel Lipodex E (50 m × 0.25 mm, |
| | 0.25 μm) |
| Oven program t/° C. | 10/120, then 1° C./min gradient to 5/120 |
| Inlet pressure | 20.9 psi, He = 1 mL/min |
| Injection température | 200° C. |
| FID température | 200° C. |
| Injection volume | 1 μL |
| Split flow | 30 mL/min |
| Diluent | MTBE |

Empagliflozin and its intermediates can be analyzed using HPLC, such as with a liquid chromatography equipped with a UV detector and the parameters described below:

| Column | Poroshell 120 EC C-18 150 mm × 4.6 mm, 2.7 μm | | | | | | |
|---|---|---|---|---|---|---|---|
| Flow | 0.8 mL/min | | | | | | |
| Load | 10.0 μL | | | | | | |
| Column oven temp | 40° C. | | | | | | |
| Detection | 225 nm | | | | | | |
| Diluent | Methanol:Water 800:200 v/v | | | | | | |
| Preparation of | Mix separately MQ water:Methanol:Acetonitrile:Ortho phosphoric | | | | | | |
| Mobile phase-A | acid in the ratio of 650:140:210:0.1 v/v/v/v. | | | | | | |
| Preparation of | Mix separately Acetonitrile:Water in the ratio of 900:100 v/v. | | | | | | |
| Mobile phase-B | | | | | | | |
| Gradient | | | | | | | |
| programme | Time (min) | 0 | 35 | 70 | 78 | 80 | 90 |
| | % M.P-A | 100 | 85 | 20 | 20 | 100 | 100 |
| | % M.P-B | 0 | 15 | 80 | 80 | 0 | 0 |

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### Definitions

The term “about” when used in the present application preceding a number and referring to it, is meant to designate any value which lies within the range of ±10%, preferably within a range of ±5%, more preferably within a range of ±2%, still more preferably within a range of ±1% of its value. For example, “about 10” should be construed as meaning within the range of 9 to 11, preferably within the range of 9.5 to 10.5, more preferably within the range of 9.8 to 10.2, and still more preferably within the range of 9.9 to 10.1.

All percentages and ratios used herein are by weight of the total composition and all measurements made are at about 25° C. and about atmospheric pressure, unless otherwise designated. All temperatures are in degrees Celsius unless specified otherwise. As used herein, “comprising” means the elements recited, or their equivalents in structure or function, plus any other element or elements which are not recited. The terms “having” and “including” are also to be construed as open ended. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example-1: Preparation of (R)-ethyl-4-chloro-3-hydroxybutyric acid ((R)-ECHB)

A 50 mL EasyMax reaction vessel was charged with NAD⁺ (10 mg), CpADH powder (40 mg, 5 wt % loading) and 100 mM pH 6.5 potassium phosphate buffer (18 mL). The EasyMax reactor system was equipped with a pH conductor, temperature probe and overhead stirring. Stirring was commenced and temperature was maintained at 20° C. A solution of ECAA (657 μL, 4.86 mmol) dissolved in 2-propanol (929 μL, 12.2 mmol, 2.5 eq.) was prepared and taken up into a syringe. The reaction was started with the addition of the substrate solution, which was slowly dosed into the reaction mixture via an external syringe pump over 14 h. The reaction was monitored by GC-FID by extracting aliquots from the reaction mixture with MTBE. When the reaction had reached completion, the mixture was filtered through a pad of Celite and then extracted with MTBE. The organic layers were dried over MgSO₄, filtered and concentrated in vacuo to yield (R)-ECHB (766 mg, 94.6% yield, >99.5% ee) as pale yellow oil.

### Example-2: Preparation of (R)-ethyl-4-chloro-3-hydroxybutyric acid ((R)-ECHB)

A 50 mL EasyMax reaction vessel was charged with NAD⁺ (10 mg), CpADH powder (50 mg, 5 wt % loading) and 100 mM pH 6.5 potassium phosphate buffer (18 mL). The EasyMax reactor system was equipped with a pH conductor, temperature probe and overhead stirring. Stirring was commenced and the reaction temperature was maintained at 20° C. A solution of ECAA (821 μL, 6.08 mmol) dissolved in 2-propanol (1.16 μL, 15.2 mmol, 2.5 eq.) was prepared and taken up into a syringe. The reaction was started with the addition of the substrate solution, which was slowly dosed into the reaction mixture via an external syringe pump over 14 h. The reaction was monitored by GC-FID by extracting aliquots from the reaction mixture with MTBE. When the reaction had stopped (93.5% conversion), the mixture was filtered through a pad of Celite and then extracted with MTBE. The organic layers were dried over MgSO₄, filtered and concentrated in vacuo to yield the crude (R)-ECHB (721 mg, 89.1% yield, >99.5% ee) as pale yellow oil.

### Example-3: Preparation of (R)-ethyl-4-chloro-3-hydroxybutyric acid ((R)-ECHB)

A 50 mL EasyMax reaction vessel was charged with NAD⁺ (15 mg), CpADH (2.44 mL from 10 g/L stock solution, 2 wt % loading) and 100 mM pH 7.0 potassium phosphate buffer (26 mL). The EasyMax reactor system was equipped with a pH conductor, temperature probe and overhead stirring. Stirring was commenced and the reaction temperature was maintained at 20° C. A solution of ECAA (985 μL, 7.29 mmol) dissolved in 2-propanol (1.39 mL, 18.2 mmol, 2.5 eq.) was prepared and taken up into a syringe. The reaction was started with the addition of the substrate solution, which was slowly dosed into the reaction mixture via an external syringe pump over 14 h. The reaction was monitored by GC-FID by extracting aliquots from the reaction mixture with MTBE. The reaction was deemed to have reached completion after 21 h, when conversion had reached 99.4%.

### Example-4: Preparation of (R)-ethyl-4-chloro-3-hydroxybutyric acid ((R)-ECHB)

A 50 mL EasyMax reaction vessel was charged with NAD⁺ (15 mg), CpADH (1.71 mL from 10 g/L stock solution, 1.4 wt % loading) and 100 mM pH 7.0 potassium phosphate buffer (26 mL). The EasyMax reactor system was equipped with a pH conductor, temperature probe and overhead stirring. Stirring was commenced and the reaction temperature was maintained at 20° C. A solution of ECAA (985 μL, 7.29 mmol) dissolved in 2-propanol (1.39 mL, 18.2 mmol, 2.5 eq.) was prepared and taken up into a syringe. The reaction was started with the addition of the substrate solution, which was slowly dosed into the reaction mixture via an external syringe pump over 14 h. The reaction was monitored by GC-FID by extracting aliquots from the reaction mixture with MTBE. The reaction was deemed to have reached completion after 21 h, when conversion had reached 97.6%.

### Example-5: Preparation of (R)-ethyl-4-chloro-3-hydroxybutyric acid ((R)-ECHB)

A 50 mL EasyMax reaction vessel was charged with NAD⁺ (10 mg), CpADH (1.63 mL from 10 g/L stock solution, 2 wt % loading) and 100 mM pH 7.0 potassium phosphate buffer (17.5 mL). The EasyMax reactor system was equipped with a pH conductor, temperature probe and overhead stirring. Stirring was commenced and the reaction temperature was maintained at 20° C. A solution of ECAA (657 μL, 4.86 mmol) dissolved in 2-propanol (929 μL, 12.2 mmol, 2.5 eq.) was prepared and taken up into a syringe. The reaction was started with the addition of the substrate solution, which was slowly dosed into the reaction mixture via an external syringe pump over 14 h. The reaction was monitored by GC-FID by extracting aliquots from the reaction mixture with MTBE. The reaction was deemed to have reached completion after 22 h, when conversion had reached 99.8%.

### Example-6: Preparation of (R)-ethyl-4-chloro-3-hydroxybutyric acid ((R)-ECHB)

CpADH (16 mg) was rehydrated in 100 mM pH 6.5 potassium phosphate buffer (1.6 mL) and 50 mM ZnSO₄ solution (32 μl) was added. The enzyme was allowed to rehydrate in the zinc/buffer solution for at least 30 min. A 50 mL EasyMax reaction vessel was charged with NAD⁺ (20 mg) and 100 mM pH 6.5 potassium phosphate buffer (17.4 mL). Following rehydration, the enzyme solution was added to the reaction vessel. Total enzyme charged into the reaction: 16 mg (2% w/w loading, 57 U). The reaction vessel was heated to 20° C. The pH of the reaction mixture was readjusted to pH 6.5 by addition of 10% K₂CO₃ solution. A substrate stock solution was prepared by dissolving ethyl-4-chloroacetoacetate 1 (657 μL, 4.86 mmol, 40 g/L substrate loading) in 2-propanol (929 μL, 12.2 mmol, 2.5 equiv.). The substrate stock solution was taken up into a 2 mL syringe and was slowly dosed into the reaction vessel over 14 hours using an external syringe pump. After 24 hours, a small aliquot (approx. 50-100 μL) of the reaction mixture was taken and extracted with MTBE (1 mL). The organic layer was separated and dried over MgSO₄ before analysis by GC-FID. When the reaction was deemed complete, MTBE (20 mL) was added to the reaction vessel. The mixture was left to stir for 10 min. The layers were separated and the aqueous phase was filtered through a pad of Celite. The Celite pad was washed with MTBE (20 mL) and the washings were subsequently used to re-extract the aqueous phase. The layers were separated and the aqueous phase was extracted with further MTBE (20 mL). The organic phases were combined, dried over MgSO₄ and concentrated in vacuo to yield the crude compound of formula II (734 mg, 91%) as yellow oil. Product attained in >99.8% conversion, >99.5% ee.

### Example-7: Isolation of CpADH Enzyme

Escherichia coli cells expressing the enzyme CpADH were obtained from a 50 L fermentation (NT2487/146) at 169 g wet cells per litre fermentation broth. A 300 g sample of cells was resuspended to 1.2 L with 50 mM potassium phosphate buffer pH 6.7 and the cell slurry was lysed by high pressure homogenization (14,000 psi, single pass) to give a crude extract (2.0 L). Zinc sulfate was added to a portion of this extract (1.0 L) to a concentration of 0.5 mM, then this material was clarified by the addition of polyethyleneimine (PEI) at a working concentration of 0.5% PEI, mixed and then centrifuged at 12,250×g for 30 minutes and 6° C. to give a clear supernatant (940 mL) containing the CpADH enzyme. This supernatant was further clarified by means of a 0.45/0.2 μm sterile capsule filter to give 920 mL of enzyme solution. This solution was then dried by means of a lyophilizer to give 20.7 g of CpADH enzyme as an off-white powder.

### Example-8: Preparation of al-tetrahydrofuran-3-ol of formula IV

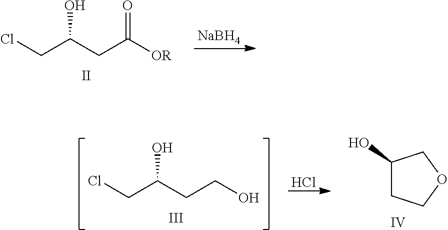

Sodium borohydride (10.5 g) and toluene (250 mL) were charged under nitrogen atmosphere into a 1000 mL round bottom flask and the suspension was cooled to 15° C. (R)-ethyl-4-chloro-3-hydroxybutyrate (prepared according to the process of example 6) (46 g, optical purity 99.5% ee) was added over 1 hour at 15° C. The mixture was stirred for 15 hours at 30° C. The reaction mixture was cooled to 5° C. and methanol (10 mL) was added slowly. The mixture was stirred for 1 hour at 30° C. Conc. HCl (30 g) and water (100 mL) were added and stirred for 15 minutes. pH was adjusted to 6.5 using 30% aqueous sodium hydroxide solution (10 mL). Layers were separated. The organic layer was washed with water and concentrated. 10% hydrochloric acid (70 mL) was added to the residue and the resulted solution was heated to 70° C. and stirred for 10 hours. The reaction mixture was cooled to 10° C. and pH was adjusted to 7.5 using 30% aqueous sodium hydroxide solution (35 mL). The reaction mixture was heated to 60° C. and extracted with ethylacetate (3×100 mL). The ethylacetate layer was concentrated under vacuum at 45° C. The residue was added to methanol (50 mL) and stirred for 15 minutes. The solution was concentrated under vacuum at 35° C. to obtain 21 g of (R)-tetrahydrofuran-3-ol. Purity by HPLC 99.3%.

### Example-9: Preparation of (R)-tetrahydrofuran-3-yl-4-methylbenzenesulfonate of compound of formula V

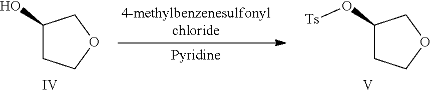

(R)-tetrahydrofuran-3-ol of formula IV (3 g), dichloromethane (45 mL) and Pyridine (10 g) were charged into a 100 mL round bottom flask and the mixture was cooled to 5° C. 4-methylbenzene-1-sulfonyl chloride (7 gm) was charged into the flask at 5° C. and the mixture was stirred for 5 hours at 28° C. Water (75 mL) was charged to the flask and stirred for 15 minutes. Layers separated and the organic layer was washed with dilute hydrochloric acid (7.5 mL of HCl in 25 mL of water). The organic layer was washed with aqueous sodium bicarbonate solution (30 mL) and water (30 mL). The organic layer was concentrated under vacuum to yield 6.8 gm of (R)-tetrahydrofuran-3-yl-4-methylbenzenesulfonate. Purity by HPLC 99.5%.

### Example-10: Preparation of Empagliflozin

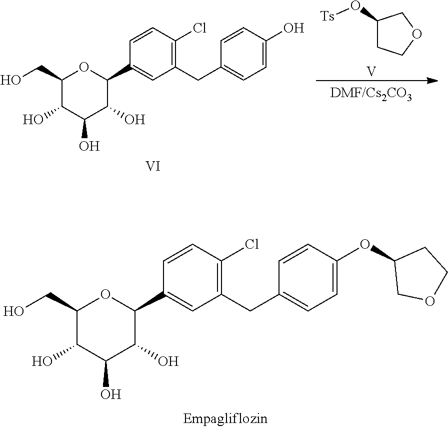

(R)-tetrahydrofuran-3-yl-4-methylbenzenesulfonate of compound of formula V (prepared in example 9) (6.34 g), compound of formula VI (10 g) and DMF (2 mL) were charged into a round bottom flask and the resulted mass was stirred for 10 minutes at 30° C. Cesium carbonate lot 1 (5.7 g) was added to the reaction mass. Reaction mass was heated to 45° C. and stirred for 2 hours at 45° C. Cesium carbonate lot 2 (5.7 g) was added to the reaction mass and the reaction mass was stirred for 2 hours. Cesium carbonate lot 3 (5.7 g) was added to the reaction mass the reaction mass was stirred for 8 hours at 45° C. The reaction mass was cooled to 30° C. and water (40 mL) was added to the mass and stirred for 10 minutes. Layers were separated and the aqueous layer was washed with toluene (40 mL). The aqueous layer was concentrated at 60° C. under vacuum until 1.0 volume remained in the reactor. The concentrated mass was cooled to 30° C. and water (100 mL) and acetonitrile 10 mL) were charged into the reactor at 30° C. and the resulted mixture was heated to 45° C. and the mixture was stirred for 3 hours at 45° C. The suspension was cooled to 25° C. and stirred for 3 hours at 25° C. The precipitation was filtered and the wet solid was washed with water (30 mL) and the solid was suck dried. The wet compound and DMF (10 mL) were charged into another flask and the solution was heated to 45° C. Acetonitrile (10 mL) charged followed by water (100 mL) into the flask at 45° C. and stirred for 2 hours. The suspension was cooled to 25° C. and stirred for 3 hours. The precipitation was fileted and the wet cake was washed with water. The wet material was suck dried. The wet material was dried under vacuum at 60° C. for 4 hours to yield 5.5 g of crystalline empagliflozin. Purity by HPLC 99.6%.

1. A process for enantioselective enzymatic reduction of keto compound of formula I 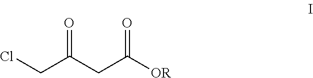
in which R represents a hydrogen atom or a C₁-5 alkyl group, comprising,
treating a mixture comprising:
(a) an (R)-specific oxidoreductase obtained from Candida parapsilosis in the range of about 5 mg to about 50 mg per gram of compound of formula I,
(b) a buffer having pH in the range of 6 to 7, and
(c) 2-propanol,
with the compound of formula I in the presence of a cofactor and zinc to form a chiral hydroxy compound of formula II

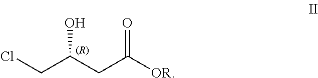

2. The process as claimed in claim 1, wherein the compound of formula I in the range of about 20 to about 100 g/L is added in to the reaction mixture gradually over the course of the reaction during the production process.

3. The process as claimed in claim 1, wherein R is ethyl.

4. The process as claimed in claim 1, wherein the compound of formula II is (R)-ethyl-4-chloro-3-hydroxybutyrate.

5. A process for preparation of (R)-tetrahydrofuran-3-yl-4-methyl benzenesulfonate of compound of formula V, comprising:
(a) reducing the compound of formula II, prepared according to the process as claimed in claim 1, using a suitable reducing agent to provide (R)-4-chlorobutane-1,3-diol of formula III 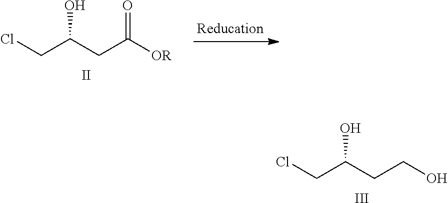
(b) cyclizing the compound of formula III to provide (R)-tetrahydrofuran-3-ol of formula IV 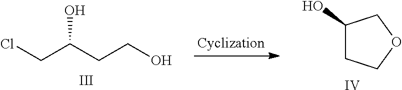
(c) converting the compound of formula IV into a compound of formula V

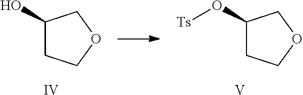

6. A process for the preparation of empagliflozin, comprising, reacting the compound of formula V prepared according to the process as claimed in claim 5 with a compound of formula VI to form empagliflozin[N]

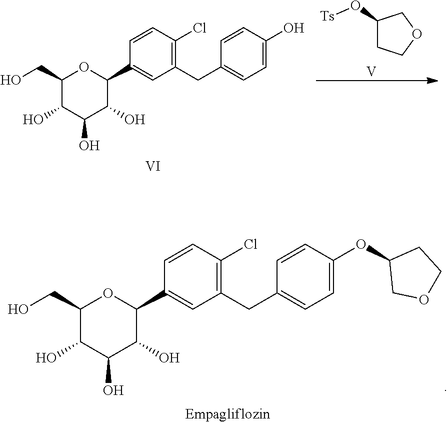

7. A process for isolation of CpADH enzyme, comprising:
(d) lysing the cells expressing the enzyme CpADH by high pressure homogenization at a pH of 6 to 7 to obtain a crude extract,
(e) adding zinc sulphate or zinc chloride to the crude extract,
(f) optionally the material is clarified by the addition of polyethyleneimine, and
(g) drying the solution using a lyophilizer to obtain the CpADH enzyme.

8. A process for enantioselective enzymatic reduction of keto compound of formula I 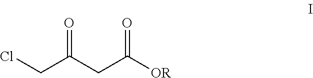
in which R represents a hydrogen atom or a C₁₋₅ alkyl group, comprising;
treating a mixture comprising:
(a) an (R)-specific oxidoreductase enzyme obtained from Candida parapsilosis, according to the process as claimed in claim 7,
(b) a buffer having pH in the range of 6 to 7, and
(c) 2-propanol,
with the compound of formula I in the presence of a cofactor and zinc to form a chiral hydroxy compound of general formula II

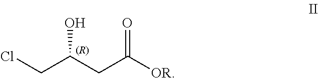

9. The process according to claim 8, the enzyme is in the range of about 5 mg to about 50 mg per gram of compound of formula I.

10. A process for the preparation of empagliflozin, comprising:
(a) reducing the compound of formula II prepared according to the process as claimed in claim 7 using a suitable reducing agent to form a compound of formula III, 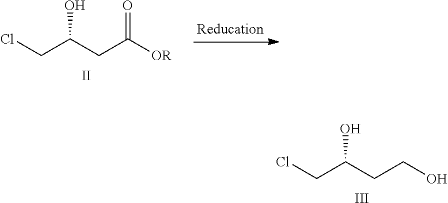
(b) cyclizing the compound of formula III to obtain a compound of formula IV, 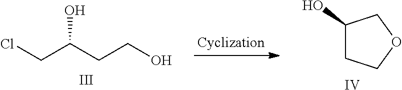
(c) converting the compound of formula IV into a compound of formula V, 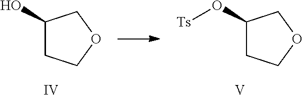
(d) reacting the compound of formula V with a compound of formula VI to form empagliflozin [chemical structure]
